(19) United States
(12) Patent Application Publication
(10) Pub. No.: US 2020/0324061 A1
(43) Pub. Date: Oct. 15, 2020

(54) Transilluminating Immobilizer for Intravenous and Intra-arterial procedures

(71) Applicant: Jacob Ament, Wausau, WI (US)

(72) Inventor: Jacob Ament, Wausau, WI (US)

(21) Appl. No.: 16/364,412

(22) Filed: Mar. 26, 2019

(51) Int. Cl. A61M 5/52; A61M 5/42

(52) U.S. Cl. CPC A61M 5/52; A61M 2210/083; A61M 2205/587; A61M 5/425

(57) ABSTRACT

A transilluminating immobilizer and methods for the same for intravenous procedures (IV), intra-arterial procedures (IA) and vessel access. The device features a component for restraining an extremity and a transilluminating component for location of vessels during IV and IA placement.

### RELATED U.S. APPLICATION DATA

Provisional application No. 62/651,636, filed on Apr. 2, 2018

### DESCRIPTION

### Field of the Invention

The present invention relates generally to methods and instruments for enhancing the performance and outcome of intravenous and intra-arterial placement. Particularly, the present invention relates to instruments that position and restrain the limb for intravenous and intra-arterial procedures (placement and/or maintenance of intravenous or intra-arterial lines) involving transillumination. More particularly, it relates to a novel device that transilluminates a limb while simultaneously locking it in the desired position during intravenous and intra-arterial procedures.

### Background of the Invention

Intravenous lines (IV) and intra-arterial lines (IA) serve a variety of important functions in hospitals and clinics. Some of these functions include administering fluids and medications to the patients. Proper placement and stabilization of these lines is necessary to ensure accurate patient monitoring and treatment. When lines are not placed or stabilized properly the fluids or medications may not properly feed into the patient's bloodstream, rendering them completely or partially ineffective. These placement and stabilization problems are often prevalent in younger patients, (infants and neonates) who lack fully developed vascular structure and sufficient cognition to exercise voluntary motor function when required. In less formal terms, they have small, difficult to locate blood vessels and don't sit still when required.

Neonates and infants often have more difficult to locate veins and arteries compared to most adult patients. Patients with darker complexion also often suffer from having more difficult to locate vessels. This can make proper IV and IA needle placement difficult. To combat this difficulty, it is often necessary to use special lights known as transilluminators to increase the visibility of veins and arteries. Current methods of transcutaneous illumination, while facilitating vein and artery location, have shortcomings that lead to major problems with “blowouts”.

Due to the fragile nature of veins and arteries, it is not uncommon for them to rupture or “blowout” because of excessive limb movement during/following IV and IA placement. Excessive limb movement can lead to shifting of the needle and/or cannula, causing it to dislodge and/or tear through vessel walls. These blowouts require that a new IV or IA placement be obtained to ensure proper treatment. Prevention of these “blown” veins and arteries requires that the limbs be held in a fixed position. To accomplish this, various devices have been developed to restrict patient movement. However, their shortcomings do not solve the problem for patients with difficult to locate vascular structure and leads to blowouts when used in conjunction with transillumination.

Due to these shortcomings, current methods and devices require multiple steps that introduce instability into the process. Thus, movement during and/or after the IV or IA has been placed causes frequent blowouts. To prevent this from occurring, the patient's limb must be immobilized and correct extension and flexion of the limb must be maintained. With current methods and devices, placing an IV or an IA requires that one must first place the transilluminator under the limb to locate a suitable vein or artery. When the best

transilluminator position is found, it must be held in place by hand. Next, the limb must be immobilized by hand. Then, while maintaining transilluminator and limb position (using one hand) the needle must be placed, which is more difficult with only one free hand. Following proper placement, the transilluminator must be removed so that the arm board can be affixed and limb immobilization reestablished. Most often, blowouts occur due to movement during the time between needle placement (transillumination) and attachment of the armboard while the limb is not immobilized. In summary, current transilluminators do not immobilize the limb or allow the limb to be immobilized properly during transillumination, nor do they ensure that immobilization is maintained. As a result, current methods are more complex, time consuming, and less stable, which results in blown veins and diminished patient outcomes. Hence, current devices are problematic and inadequate for neonates, infants, and other patients requiring transillumination for IV procedures.

Various devices have been developed for transillumination and immobilization but none stabilize the limb for the transillumination process and are easily detachable from the base for cleaning, repositioning, imaging (MRI, CT, X-ray, etc. . . . ) and/or maintenance. Therefore, these devices, that accomplish the tasks of transillumination and immobilization, fall short in overall utility, and are completely useless in many situations. Often times a patient may require imaging (CT scans, X-Rays, MRIs, etc). Therefore, it is important that the device used to transilluminate be compatible with imaging techniques in addition to maintaining immobilization throughout the entire treatment process. In previous devices, incompatible parts cannot be separated from the device in a timely manner without disassembly or removal of the device from the patient. Removal of the device means loss of extremity immobilization, thereby increasing the chance of blowout and treatment failure, requiring that the IV or IA placement be repeated and a new insertion site identified. As a result, current devices are inadequate for patients requiring these procedures.

Eliminating excess movement from patients undergoing IV and IA therapies will reduce the number of blowouts patients experience. Reducing or eliminating blowouts would not only improve patient comfort, lessen pain, suffering, and tissue damage by reducing the number of needle sticks, but it would also increase patient safety. By reducing blowouts, the number of placement attempts would be reduced, which would mean patients will receive fluids and/or medication sooner. In an emergency, timely administration of fluids or medication can be the determining factor in a patient's survival. Therefore, what is needed; is a method and device that saves lives and improves both patient comfort and safety by reducing the chance of vessel blowout and minimizing procedure time.

It would be desirable to provide a mobile device that is capable of maintaining extremity immobilization prior to, during, and after vessel transillumination and throughout treatment in an uninterrupted manner.

Additionally, it would be desirable to provide a device that eliminates the need for handheld transillumination comprising readily removable electronic components affording use in conjunction with the various forms of imaging and other common medical procedures.

It would further be desirable to provide an device that can immobilize the limb and adjusts for varying postures, (as is often required for finding insertion sites due to the varying anatomy of patients) while affording transillumination to the patient's limb during IV and IA therapy.

### SUMMARY AND OBJECTS OF THE INVENTION

Described herein are various embodiments of a transilluminator that allows health care workers and/or veterinarians to position, immobilize, and maintain the desired posture of a patients limb or extremity in an uninterrupted manner throughout IV or IA treatment (prior to, during, and after needle placement).

Accordingly, it is an object of this invention to provide an instrument and method for the instrument's use that simplifies IV and IA procedures that involve transillumination.

It is also an object of this invention to provide an instrument that increases the ease with which a health care worker can achieve and maintain an IV or IA therapy and reduces the time required to achieve and maintain an IV or IA.

It is a further object of this invention to provide a device that reduces the frequency of blown arteries, veins, and vessels.

It is a still further object of this invention to provide an adjustable instrument that can immobilize a patient's limb and provide simultaneous transillumination to the immobilized limb.

It is a still further object of this invention to provide a device with detachable transilluminating component that is compatible and can be used in conjunction with medical imaging procedures (MRI, CT, X-ray, etc. . . . ). Furthermore, the transilluminating component can be detached for repositioning, cleaning, and maintenance.

In accordance with the above objects of the invention the applicant describes an instrument suitable for Intravenous and Intra-arterial procedures. The instrument has a member or members for affixing the instrument to a patient's extremity. The instrument is capable of adjustable transillumination and adjustable immobilization to the patient's extremity.

The device includes an adjustable extremity immobilizing instrument and lights of varying and/or variable wavelengths with adjustable intensity to illuminate subcutaneous tissue. Additionally, a clear, disposable, protective cover protects the device, providing a barrier from fluids and contaminants and prevents the spread of infection when desired.

Accordingly, it is the principal object of this invention to provide a new device for locating veins and/or arteries with transillumination while simultaneously immobilizing patient extremities and in an uninterrupted manner, thereby eliminating the problems that result from loss of extremity immobilization.

### BRIEF DESCRIPTION OF THE DRAWINGS

The aforementioned and further objects, forms, features, and advantages of the present invention are made apparent from the subsequent description of the drawings where like reference characters denote embodiments of like elements throughout the various views.

FIG. 1A is a plan view from the bottom of an embodiment of the overall device where straps affix the device to the underside (palm side) of the limb to stabilize the wrist and the light is directed into the palm side through a transparent immobilizing component and through the limb to transilluminate and locate vessels on the distal portion of the top side of the limb.

FIG. 1B Is a plan view from the top of an embodiment of the overall device where straps affix the device to the underside (palm side) of the limb to stabilize the wrist and the light is directed into the palm side through the limb to transilluminate and locate vessels on the top side of the limb.

FIG. 1C is a plan view of an embodiment of the overall device as shown in FIG. 1A-1B where the transilluminating component has been positioned to transilluminate a proximal portion of the limb.

FIG. 1D is a side view of an embodiment of the overall device as shown in FIG. 1A-1B where the transilluminating component is separated from the base when desired (cleaning, maintenance, patient imaging, or repositioning).

FIG. 2 is a perspective view of an embodiment of the overall device where the device is attached to the leg/foot instead of the arm/hand and a detachable power source is housed separately from the transilluminating component.

FIG. 3 is a side view of an embodiment of the overall device where the device is attached to the extremity of a veterinary patient (animal) instead of a human patient.

FIG. 4 is a perspective view from behind a patient (dorsal side) of an embodiment of the overall device where the device is being used to stabilize the elbow joint instead of the wrist joint while transilluminating vessels on the inside of the elbow/arm.

FIG. 5 is a perspective view of an embodiment of the overall device where flexible or jointed clamping arms, instead of straps, affix the device to the extremity.

FIG. 6 is a plan view from the bottom of the overall device where a malleable material is used for the framework of the base to adjust the shape and light passes through openings in the base.

FIG. 7 is a side view of an embodiment of the overall device where the device features an adjusting mechanism for adjusting the angle of the light waves relative to the immobilizing component and where the transilluminating component affixes to the immobilizing component along a groove on the outside edge.

FIG. 8 is a cross-sectional view taken along line 8-8 of FIG. 7.

FIG. 9 is a top view of an embodiment of the overall device where the transilluminating component of the device is attached to the same surface as the desired insertion site and uses lateral transillumination instead of transilluminating through the entire diameter of the extremity.

FIG. 10A is a cross-sectional view taken along lines 10-10 of FIG. 4 where a flexible transilluminating component is separate from a surface and in combination with FIG. 10B demonstrates how a flexible transilluminating component conforms to the shape of a surface upon which it is placed.

FIG. 10B is a cross-sectional view taken along lines 10-10 of FIG. 4 where a flexible transilluminating component is laying upon a surface and in combination with FIG. 10A demonstrates how a flexible transilluminating component conforms to the shape of a surface upon which it is placed. FIG. 11A is a front side view of an embodiment of an adapter for use with an immobilizing component like the one shown in FIG. 7.

FIG. 11B is a perspective view of the adapter illustrated in FIG. 11A. FIG. 11C is a perspective view of the adapter illustrated in FIG. 11A.

FIG. 12A is a perspective view of an embodiment of the transilluminating component wherein protuberances on the housing affix the component to an immobilizer.

FIG. 12B is a perspective view of an embodiment of the immobilizing component for use with the transilluminator in FIG. 12A.

FIG. 12C is a perspective view of an embodiment of the overall device where FIGS. 12A and 12B are affixed to each other.

FIG. 13A is a perspective view of an embodiment of the transilluminating component with suction cup affixing means.

FIG. 13B is a perspective view of an embodiment of the immobilizing component for use with the transilluminator in FIG. 13A.

FIG. 14 is a perspective view of an embodiment of the immobilizing component with adhesive affixing means.

FIG. 15A is a perspective view of an embodiment of the immobilizing component featuring a fluid bladder in an empty state.

FIG. 15B is a perspective view of an embodiment of the immobilizing component featuring a fluid bladder in a filled state.

### DETAILED DESCRIPTION OF THE PREFERRED EMBODIMENTS

### A. General Information

The use of the terms “a,” “an,” and “the” and similar references in the context of this disclosure (especially in the context of the following claims) are to be construed to cover both the singular and the plural, unless otherwise indicated herein or clearly contradicted by context. All methods and individual method steps described herein can be performed in any suitable order or simultaneously, unless otherwise indicated herein or otherwise clearly contradicted by context. The use of any and all examples, or exemplary language (e.g., such as, preferred, preferably) provided herein, is intended merely to further illustrate the content of the disclosure and does not pose a limitation on the scope, or range of equivalents, to which the appended claims are entitled. No language in the specification should be construed as indicating any non-claimed element as essential to the practice of the present disclosure.

All references, including printed publications, patent applications, and patents, cited herein are hereby incorporated by reference to the same extent as if each reference were individually and specifically indicated to be incorporated by reference and were set forth in its entirety herein.

Alternative embodiments of the claimed disclosure are described herein. Of these, variations of the disclosed embodiments will become apparent to those of ordinary skill in the art upon reading the foregoing disclosure. The inventors expect skilled artisans to employ such variations as appropriate (e.g., altering or combining features or embodiments), and the inventors intend for the embodiments to be practiced otherwise than as specifically described herein.

Accordingly, the embodiments include all modifications and equivalents of the subject matter recited in the claims appended hereto as permitted by applicable law. Moreover, any combination of the elements disclosed herein in all possible variations thereof is encompassed by this disclosure, unless otherwise indicated herein or otherwise clearly contradicted by context.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within

the ranges. As used herein, the terms “about” and “approximately,” when referring to a numerical value, shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors that may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Thus, recitation of ranges of values herein are merely intended to serve as a shorthand method of referring individually to each separate value falling within the range, unless otherwise indicated herein, and each separate value is incorporated into the specification as if it were individually recited herein.

It is to be understood that any ranges, ratios, and ranges of ratios that can be formed by, or derived from, any of the data disclosed herein represent further embodiments of the present disclosure and are included as a part of the disclosure as though they were explicitly set forth. This includes ranges that can be formed that do or do not include a finite upper and/or lower boundary. Accordingly, a person of ordinary skill in the art most closely related to a particular range, ratio, or range of ratios will appreciate that such values are unambiguously derivable from the data presented herein.

“Affixing means,” as used herein, includes, but is not limited to, methods and/or materials for attaching one object or thing to another.

“Base,” as used herein includes, but is not limited to, any part(s) of the device that, when used in combination with an affixing means secures the overall device to the extremity and inhibits the extremity's movement. Additionally, a “base” includes any part of the immobilizing component or adapter component to which any portion of another component may be affixed.

“Bottom,” as used herein to refer to the immobilizer, includes, but is not limited to, the surface of the immobilizer that faces away from the extremity.

“Contoured,” as used herein, includes, but is not limited to, molded or shaped to fit a certain configuration. “Contoured” additionally includes, but is not limited to, any shape that provides comfort to a patient. “Contoured” further includes, but is not limited to, bendable or shaped to fit a patient at the time of application.

“Control,” as used herein, includes, but is not limited to a switch or other device by which the transilluminating component is adjusted and/or regulated.

“Corner,” as used herein, includes, but is not limited to, a place where two converging sides meet at the vertex of an angle.

“Extremity,” as used herein, includes, but is not limited to, a limb or portion of a limb, such as a leg, ankle, foot, toe, arm, wrist, hand, finger, thumb, neck, head, wing, tail, claw, paw, hoof, or fin.

“Hands-free,” as used herein, includes, but is not limited to, methods and/or materials designed to be used without being held in the hands.

“Hinge,” as used herein, includes, but is not limited to, any object or component of the present invention that provides a movable joint or mechanism that connects linked objects. Examples include hinges similar in construction and/or design to those used on many household doors (which is movable along one axis) or a ball and socket style joint (which is movable along multiple axis).

“Immobilizer” or “immobilizing component,” as used herein, includes, but is not limited to, any material or object that an extremity may be braced against, so as to inhibit the extremity's movement at one or more joints.

“Insertion site,” as used herein, includes, but is not limited to, any wound, opening, or lesion in the skin, or more than one wound, opening, or lesion, such as those made by needles and/or those made for peripheral or midline catheters, central venipuncture venous access catheters, or peripherally inserted central catheter (PICC) line. “Insertion site” may also include accompanying equipment—for instance, equipment present at an intravenous site, such as an IV catheter, extension tubing, Luer-Lok™ tubing, a loop of tubing, a catheter, locking mechanism (e.g., Luer-Lok™), extension tubing, transparent dressing, securement devices, tape, or wound, opening, and lesion dressing materials such as gauze.

“Leg support,” as used herein, includes, but is not limited to, a device that stabilizes the leg and/or the foot.

“Light,” as used herein, includes but is not limited to, a device that provides electromagnetic waves through the use of light emitting diodes (LEDs), bulbs, halogen, fluorescent, incandescent or other types of lighting that may be available, along with the associated controls, circuitry, and/or electronics required for the proper function of said device. “Light” further includes, but is not limited to, electromagnetic waves.

“Locking,” as used herein, includes but is not limited to, any object or component that limits or prevents the movement of a hinge.

“Means for closing,” as used herein, includes, but is not limited to, a method and/or material to fasten objects together.

“Palm,” as used herein, includes, but is not limited to, the part of the ventral surface of the hand that extends from the wrist to the bases of the fingers.

“Patient,” as used herein, includes, but is not limited to, a human or other animal that needs to have an extremity immobilized.

“Plastic,” as used herein, includes, but is not limited to, polystyrene, polycarbonate, acrylics, silicone rubber, polyethylene, polypropylene, polyester, polyimide, and synthetic rubbers. “Plastic,” as used herein, also includes plastic resins, for example, polypropylene homopolymer. Further, as used herein, “polypropylene” includes, but is not limited to, oriented polypropylene and biaxial oriented polypropylene. As used herein, “polycarbonate” includes, but is not limited to, brands such as Lexan®, Makrolon®, or Makroclear®. As used herein, “polystyrene” includes, but is not limited to, Styrofoam®. As used herein, “acrylics” include, but are not limited to, those sold under trade names such as Lucite®, Optix®, Perspex®, Altuglas®, or Plexiglass®. As used herein, “polyethylene” includes, but is not limited to, high-density polyethylene, low-density polyethylene, ethylene vinyl acetate, or polyvinyl chloride. As used herein, “polyester” includes, but is not limited to, semi-crystalline polyethylene terephthalate, amorphous polyethylene terephthalate, recycled polyethylene terephthalate, or polyethylene terephthalate glycol. As used herein, “synthetic rubbers” include, but are not limited to, polychloroprene or polyisoprene.

“Power source,” as used herein, includes, but is not limited to, and object or material that provides and/or transmits electricity to the transilluminating component. This includes batteries (electrochemical cells), any type of capacitor, and any other means by which electrical charge can be provided or stored.

“Rectangular,” as used herein, includes, but is not limited to, a polygon with four sides and four corners. “Rectangular” additionally includes, but is not limited to, one to four rounded corners and/or one to four rounded sides.

“Rounded corner,” as used herein, includes, but is not limited to, a side that bends without an angle. “Rounded corner” additionally excludes a vertex where straight sides meet.

“Rounded side,” as used herein, includes, but is not limited to, a side that does not form a straight line. “Rounded side” additionally includes, but is not limited to, a side that is concave, convex, a mixture thereof, or straight, concave, and/or convex.

“Strap,” as used herein, includes, but is not limited to, one or more pieces of material that brace or secure an immobilizer to an extremity.

“Transilluminating component,” as used herein, includes, but is not limited to a light, a power source, controls, and/or any object or component of the present invention that provides transillumination.

“Transparent,” as used herein, includes, but is not limited to, any material or object that transmits rays of light through its substance so that bodies situated beyond or behind can be distinctly seen. “Transparent” also includes, but is not limited to, easily seen through. Further, “transparent,” as used herein, includes, but is not limited to, any material or object wherein a portion of that object is see-through, regardless of whether the remaining portion of that object or material is cloudy or opaque.

“Window,” as used herein, includes, but is not limited to, one or more openings that allow visualization and/or palpation of the extremity.

A “window” further includes at least one transparent portion. Additionally, a “window” includes one or more areas that do not impede visualization and/or palpation of the extremity.

### B. Description

10. Immobilizing component
11. Transilluminating component
13. Affixing means: device to patient
14. Hinge
15. Locking mechanism
16. Light Emitting Diode (LED)
17. Housing for power source and controls
18. Power Cord
19. Light to Base Mount
20. Vessel
21. Base
22. Transilluminator Housing
23. Adjustment knob
24. Affixing means: transilluminator to immobilizer
25. Cut-out window
26. Groove for affixing transilluminator to immobilizer
27. Rail for affixing
28. Adapter base
29. Affixing means, attachment to adapter
30. Hinge for adjusting the direction of light
31. Mortise
32. Tenon
33. Tab
34. Suction placement surface
35. Adhesive patch
36. Inflatable bladder
37. Suction cup
38. Clamping screw
39. Adjustment plate
100. Arm support
200. Leg support
300. Limb support

Because embodiments may take various forms, the description below of several embodiments is made with the understanding that the present disclosure is to be considered as an exemplification of the claimed subject matter, and is not intended to limit the claims to the specific embodiments illustrated and/or described. The headings used throughout this disclosure are provided for convenience only and are not to be construed to limit the claims in any way. Embodiments illustrated under any heading may be combined with embodiments illustrated under any other heading.

The various embodiments disclosed and described herein present a device that comprises an immobilizing component 10, a transilluminating component 11, affixing means 13, and a power source with controls 17. In some embodiments, the immobilizing component 10 comprises a single part of a fixed shape. In other embodiments the immobilizing component 10 comprises a single part of adjustable shape. Still other embodiments of the immobilizing component comprise multiple parts of fixed shape. Yet other embodiments of the immobilizing component comprise multiple parts of adjustable shape.

An immobilizing component 10 and affixing means 13 can be constructed using any material that is compatible with X-ray, MRI, and CT imaging. Specifically radiographically, tomographically, and fluoroscopically non-disruptive materials, non-ferromagnetic materials, or any combination thereof. Materials can include, but are not limited to plastics, woods, composites, non-disruptive metals and alloys, and any mixture thereof. An affixing means can also be constructed of any non-ferromagnetic material. Materials can include but are not limited to adhesives, plastics, woods, composites, aluminums, titaniums, brass, copper, or any mixture thereof. It will be understood, by those skilled in the art, that the immobilizing component 10 can comprise various shapes that all immobilize the extremity effectively. Shapes can include, but are not limited to flat, curved, rectangular, round, hourglass, cylindrical, solid, hollow, or any combination thereof. Furthermore, it will be obvious to those skilled in the art that the form, shape, and/or contours of embodiments of an immobilizing component may vary.

In one embodiment, the present invention provides immobilization by affixing to an arm. In another embodiment, the present invention provides immobilization by attaching to a leg. In one embodiment, the immobilizing component 10 is a frame with an opening through which the transilluminating component 11 can transmit light. In another embodiment, the immobilizing component 10 has a transparent portion through which the transilluminating component 11 can transmit light.

One embodiment of the transilluminating component 11 features light emitting diodes (LEDs) 16 and a built-in power source with controls 17. In another embodiment, the transilluminating component 11 has a separately-housed power source with controls 17. In still another embodiment, the transilluminating component 11 has a detachable power source with controls 17. The transilluminating component 11 may be adjustable with regard to wavelength and intensity of the light transmitted. This may be accomplished using a light of variable wavelength or a plurality of selectable lights of different wavelengths.

A further embodiment of the present invention comprises an immobilizing component 10, power source with controls 17, and a transilluminating component 11, wherein the immobilizing component 10 comprises a base 21 with means 13 for affixing the device to the patient. In one embodiment, the affixing means is a strap and a means for closing the strap around a limb. In another embodiment, the affixing means 13 is a clamp or base extension for clamping the base to a limb. In yet other embodiments, the affixing means 13 by which the device is affixed to a patient can include, but are not limited to, adhesive strips, elastic straps, flexible latching straps and any other means familiar to those skilled in the art.

Still further embodiments of the invention additionally comprise various adapters to which a transilluminator or other device of different make or model may be affixed. Once affixed to said adapter, the adapter may then be affixed to the immobilizing component 10.

### C. Examples

The accompanying figures illustrate several embodiments of an extremity immobilizing transilluminator. Arm 100, leg 200, and animal limb 300 immobilizing transilluminators are exemplified. It will be obvious to those skilled in the art, that the disclosed embodiments may vary in size, shape, and design to accommodate the utilization of each embodiment for use with all types of extremities (arm, leg, hand, animal limb, paw, fin, tail, wing).

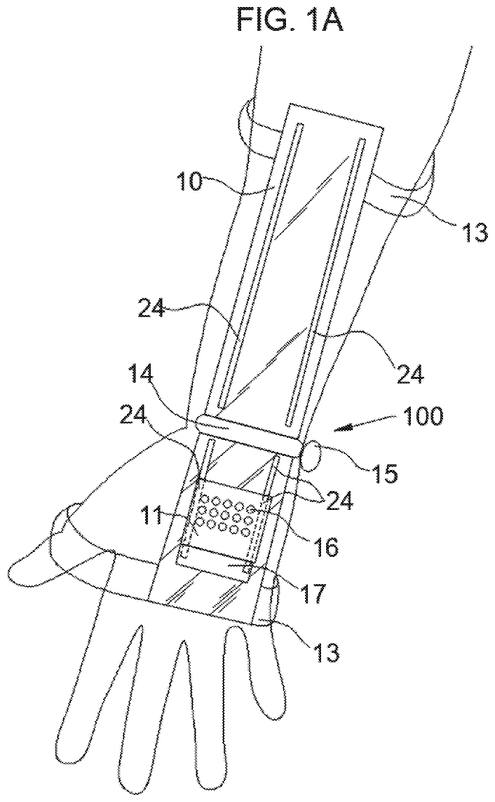

FIGS. 1A-1D disclose examples of an arm support comprising flat, transparent, rectangular immobilizing components 10, a repositionable transilluminating component 11, and affixing means 13 for affixing the device to a human arm. The transilluminating component 11 has a built in power source and controls 17. An additional affixing means 24 attaches the transilluminating component 11 to the immobilizing component 10 in a manner that affords repositioning along the immobilizing component 10. The affixing means 24 wherein the transilluminating component 11 affixes to the immobilizing component 10 can comprise various materials or designs that allow for quick, easy detachment, repositioning, and/or adjustment of the transilluminating component 11, including, but not limited to hook-and-loop fasteners, adhesive strips, removable magnetic strips, buckles, negative air pressure devices (suction cups or other vacuum devices), a sliding rail/track/groove system or a system of complementary pegs/holes, groove(s), or protuberances utilizing friction (think Lego®-style building blocks). The design allows repositioning and/or detachment of the transilluminating component 11 without disassembly of the components. The transilluminating component 11 can be repositioned to various sites along the immobilizing component 10 as demonstrated by the different positions illustrated in FIG. 1A and FIG. 1C to achieve transillumination of different portions of the extremity. In this embodiment a hinge 14 mechanism allows for angle adjustment of the immobilizing component 10 for the purposes of achieving a patient posture conducive to placement and secure maintenance of an IV. A locking mechanism 15 for the hinge 14 maintains the desired angle once achieved.

In another embodiment, the immobilizing component 10 is not comprised entirely of a transparent material, and instead has cutouts that may be left open or filled with a transparent material that act as windows to allow light to pass into the extremity. It will be obvious to those skilled in the art, that the locking mechanism may vary, including but not limited to a quick release cam clamping devices, clamping screws, and hirth joints. In other embodiments, the immobilizing component 10 comprises curved surfaces that mimic the shape of the patient's extremity. FIG. 1D illustrates the embodiment as illustrated by FIGS. 1A-1C in a state where the transilluminating component 11 is separated from the immobilizing component 10. FIG. 1D also illustrates a the path of movement afforded by the hinge 14 for the angle adjustment of the immobilizing component. Light passes through the entire diameter of the patient's limb from the transilluminating component 11 to aid needle

placement on the surface of the patient that is opposite the surface that is in contact with the transilluminating component 11. In FIGS. 1A-1C, the device is shown mounted to the palm side of the arm to transilluminate vessels and locate insertion sites on the back side of the arm.

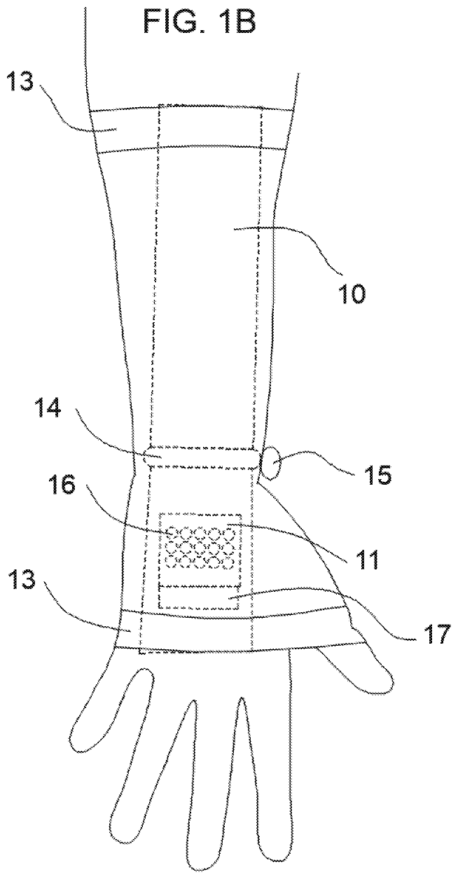

FIG. 2 is a plan view of an embodiment of a leg immobilizing transilluminator 200 wherein immobilization of the ankle is achieved by affixing two parallel, rigid, adjustable, pieces of the immobilizing component 10 to a patient's leg using straps 13. The pieces of the immobilizing component 10 feature locking hinges 14 to adjust and maintain the desired patient posture and angle of immobilization. The transilluminating component 11 features affixing means 19 whereby the transilluminating component 11 is affixed to the immobilizing component 10 in a manner allowing the transilluminating component 11 to be repositioned to various places along the immobilized portion of the patient's leg. In an embodiment illustrated by FIG. 2, the power source with controls 17 is housed separately from the transilluminating component 11 and connected through means of a detachable power cord 18. Light passes through the patient's limb from the transilluminating component 11 to aid needle placement on the surface of the patient that is opposite the surface that is in contact with the transilluminating component 11. It will be obvious to those skilled in the art, that a power source may be integrated into a transilluminating component 11 and the controls housed separately. It will further be obvious that signals transmitted through a cord 18 may be transmitted remotely with controls utilizing wireless technology. This technology includes but is not limited to various electromagnetic wave transmission technologies such as infrared light (like TV remotes), Bluetooth technology, and WiFi. In these embodiments, the cord 18 takes the form of an electromagnetic wave that transmits a signal which is received by the transilluminating component 11 to control wavelength and intensity.

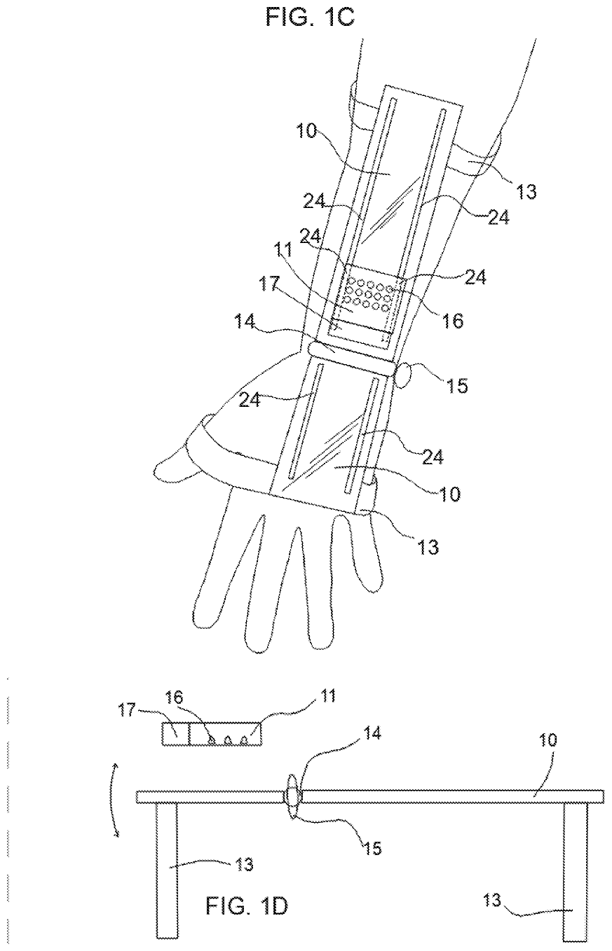

FIG. 3 is a side view of the side of an animal limb immobilizing transilluminator 300 of an embodiment similar in overall design to the examples illustrated by FIGS. 1A-1D wherein the device is affixed to a non-human (animal) patient. It comprises a repositionable transilluminating component 11 with LEDs 16, an immobilizing component 10 with locking hinge 14 and means for affixing it to a transilluminating component 11, and affixing means 13 for affixing the device to a patient's extremity.

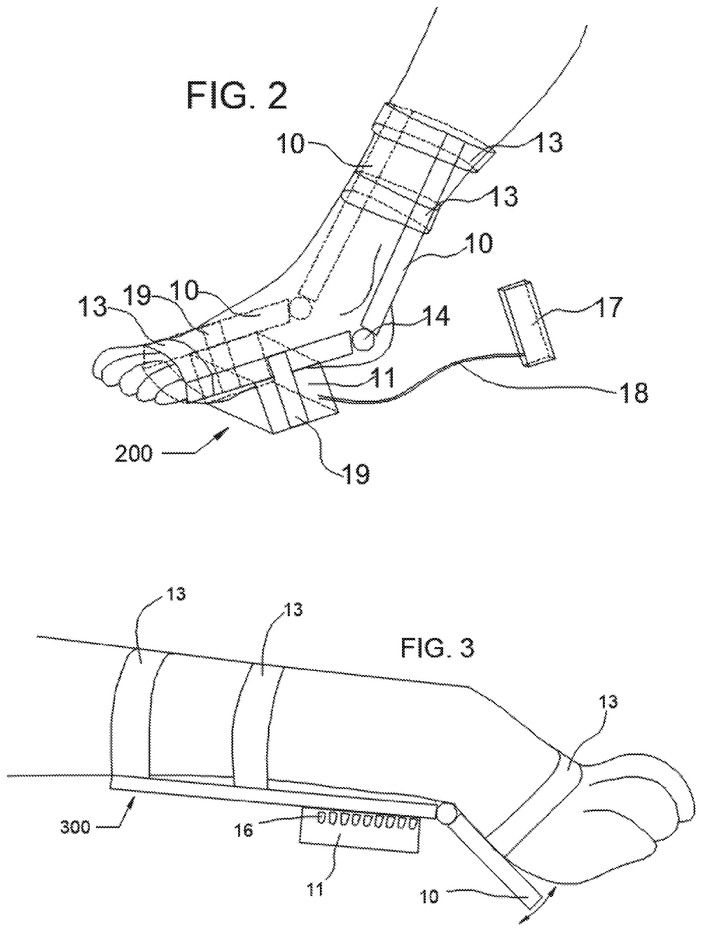

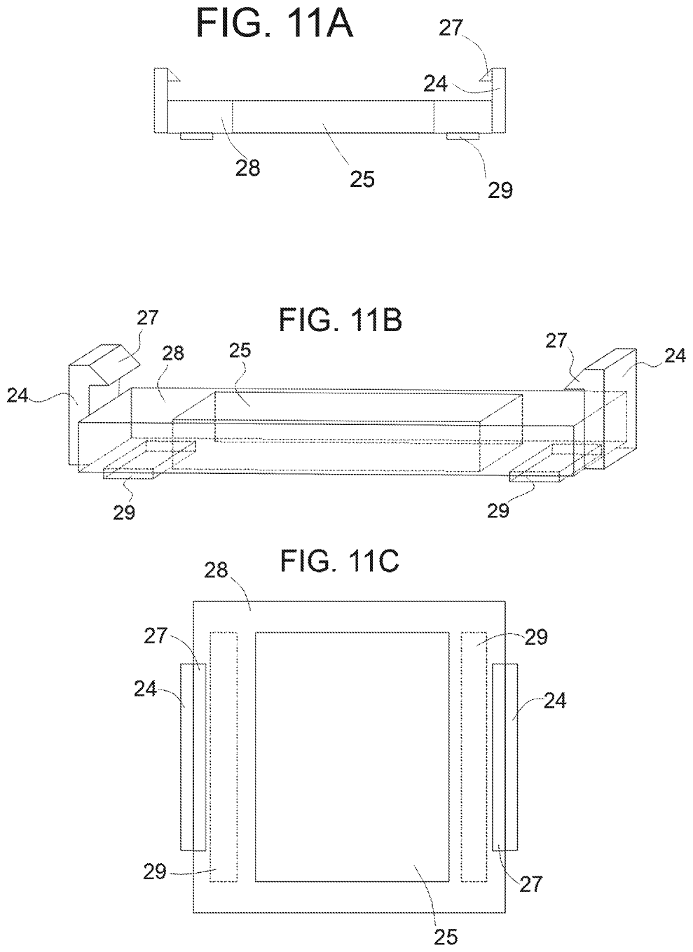

FIG. 4 discloses an arm immobilizing transilluminator 100 wherein immobilization of the elbow joint is achieved with a curved, immobilizing component 10 and straps 13. The base of the immobilizing component 10 is contoured to more comfortably fit the natural curvature of a patient's extremity and features a locking hinge 14 by which the angle of the immobilizing component 10 may be adjusted to achieve and maintain a desired extremity posture. A flexible, removable, and repositionable, transilluminating component 11 is illustrated with separately housed power source 17 connected by detachable power cord 18. The controls for the transilluminating component 11 can be housed with the transilluminating component 11, the power source 17, or separately with either a wired or wireless remote control. In this configuration, light is directed into the dorsal surface of the extremity, passes through the extremity, and transilluminates vessels on the ventral side for location of an insertion site. FIG. 10 illustrates how a flexible transilluminating component 11 with LEDs 16 flexes to match the contours of the surface upon which it is placed; which, as shown in FIG. 10, is a transparent immobilizing component 10.

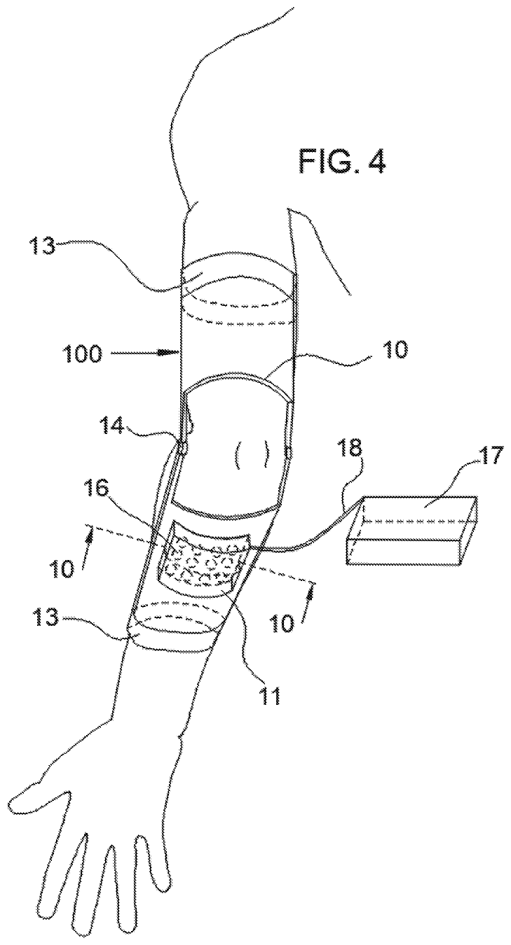

FIG. 5 discloses an embodiment of the overall device comprising a transparent immobilizing component 10 with a locking hinge 14, a repositionable transilluminating component 11, and affixing means 13 in the form of curved flexible clamping arms that affix the immobilizing component 10 to a patient's extremity. In a similar embodiment, the clamping arms 13 have locking hinges, instead of flexible clamping arms, to adjust the fit to a patient's extremity.

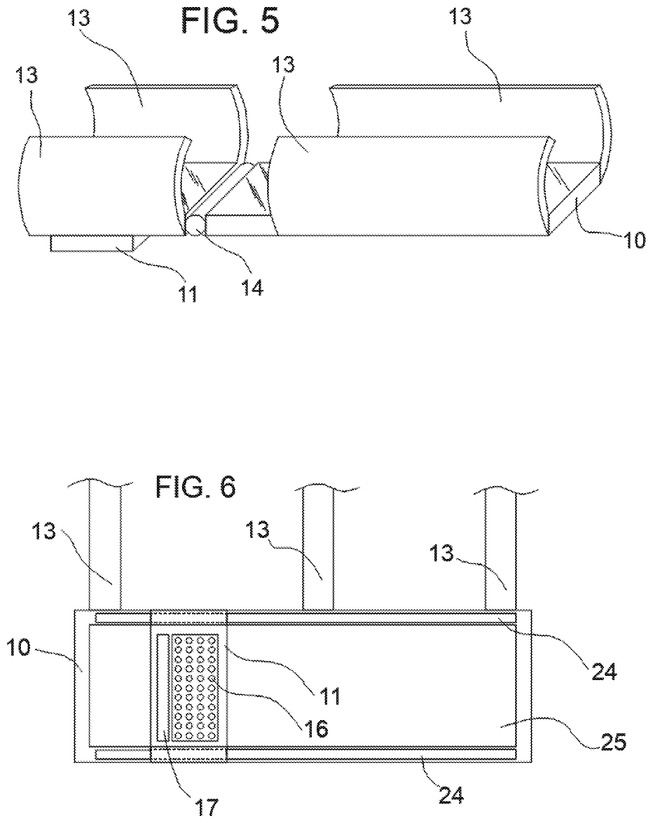

FIG. 6 is a plan view from the bottom of an embodiment of the overall device comprising an immobilizing component 10 with means 24 for affixing it to a transilluminating component 11, a repositionable transilluminating component 11 with LEDs 16, and affixing means 13 for affixing the device to a patient's extremity. In this embodiment, FIG. 6 illustrates an immobilizing component 10 that is comprised of a malleable frame with a window 25 cut out allowing for transillumination of the patient's extremity. The malleable material affords the ability to adjust the immobilizing component 10 to a desired shape for immobilization. Affixing means 24 for affixing the transilluminating component 11 to the immobilizing component 10 are located along the length of the immobilizing frame 10 affording repositioning of the transilluminating component 11 along the length of the device.

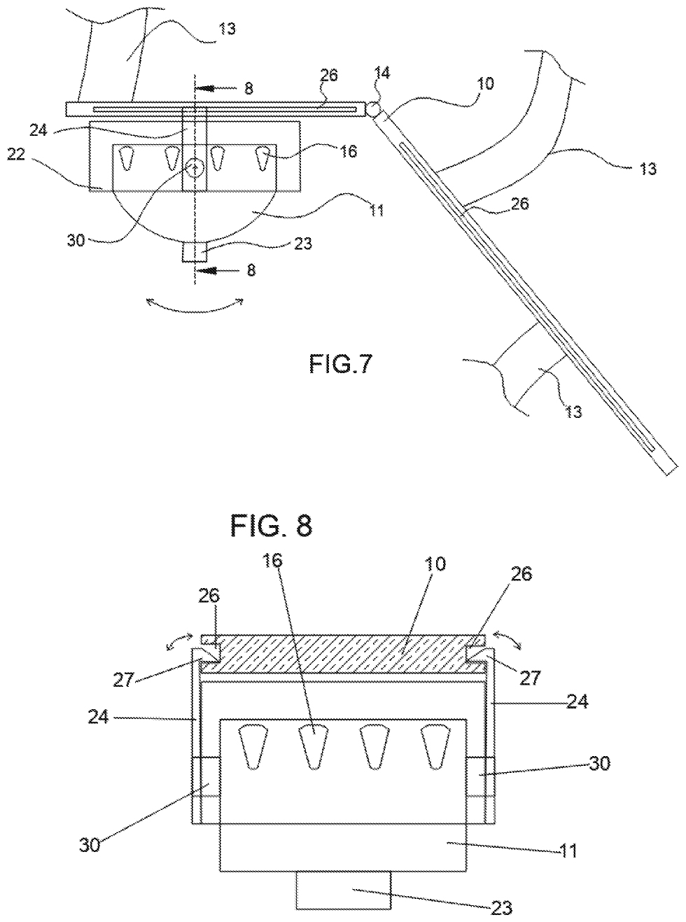

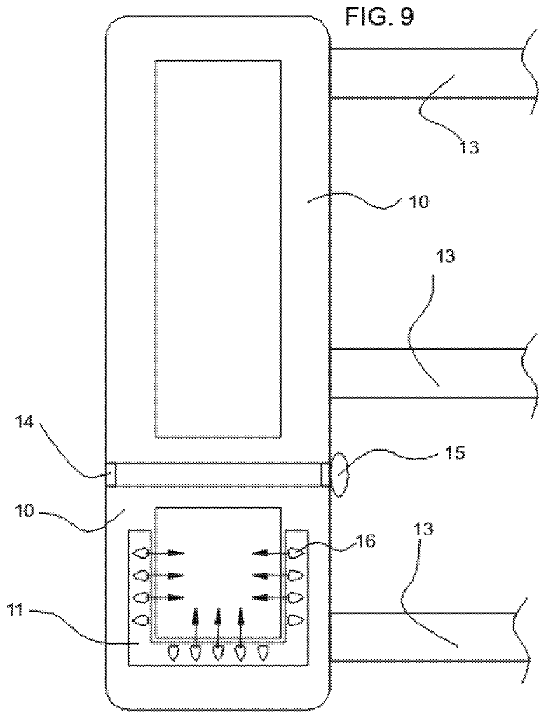

FIG. 7 discloses a side view from the side of an embodiment of the overall device comprising a transparent immobilizing component 10 with a locking hinge 14 and means for affixing it to a transilluminating component 11, an adjustable, repositionable transilluminating component 11 with LEDs 16, and affixing means 13, in the form of straps, for affixing the device to a patient's extremity. A groove 26 is illustrated along the length of the immobilizing component 10 for the purposes of mating with affixing means 24 on the transilluminating component 11. The cross section of line 8-8 from FIG. 7 that is illustrated in FIG. 8 further clarifies one embodiment of this affixing means 24. In this embodiment, the direction of the light is adjusted by means of a hinge 30 located on the transilluminating component 11. In other embodiments, a malleable affixing means 24 may bend to adjust the direction of light transmission. Manipulation of an adjustment knob 23 pivots the lights in relation to the transilluminator housing 22.

FIG. 8 shows a cross section of an embodiment similar to FIG. 7 comprising a transparent immobilizing component 10 with a groove 26 on the edges. A beveled rail 27 is located at the end of affixing means 24 on each side of the transilluminating component 11. The rails 27 clip into a groove 26 on each side to mate the transilluminating component 11 to the immobilizing component 10. A hinge 30 for adjusting the direction of light rotates the transillouminating component 11. Sufficient friction holds the transilluminating component 11 in place preventing undesired movement. However, when desired, the affixing clip 24 can slide along the grooves 26 for repositioning.

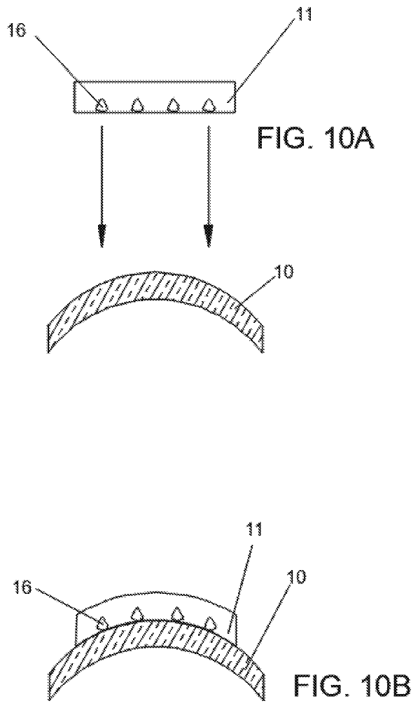

FIG. 9 discloses an embodiment of the overall device comprising a hinged 14, rectangular immobilizing component 10 with locking mechanism 15, affixing means 13 in the form of straps, and a lateral transilluminating component 11. The immobilizing component 10 features rounded corners. Arrows indicate the general direction of light transmission relative to the device. In this embodiment, the transilluminating component 11 transilluminates laterally rather than perpendicular to the surface of the extremity as has been disclosed in the previous embodiments. This method is useful for location of an insertion site on the same surface as the transilluminating component 11. For example, if the device is mounted to the palm side of a patient's arm it transilluminates vessels on the palm side of the patient's arm. This is in contrast to the perpendicular transillumination disclosed in the last sentences of the paragraph describing FIG. 1A-1C, wherein a device mounted to the palm side transilluminates vessels on the back side of the arm.

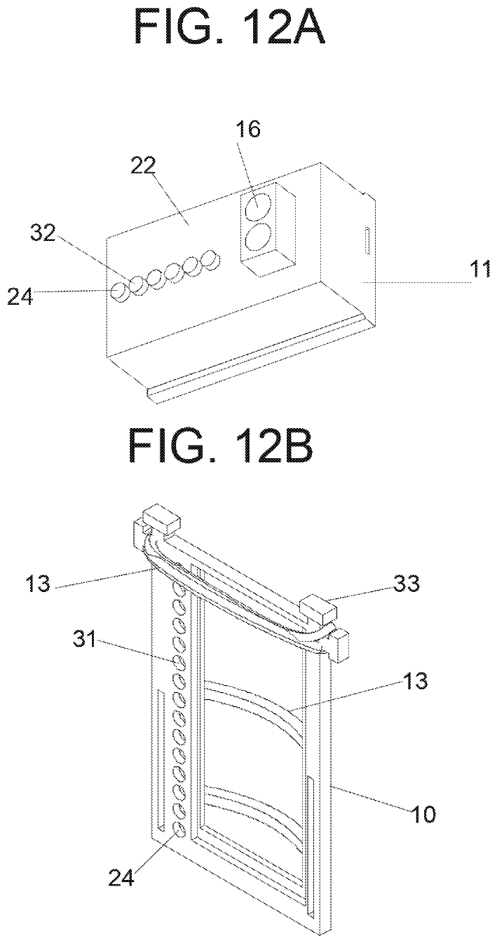

FIGS. 11A-11C disclose an embodiment of an adapter component with a window 25 cut out from the base 28. The adapter is used to connect alternate attachments and accessories to the overall invention. In this embodiment, the affixing means 24, whereby the adapter is affixed to the immobilizing component use a rail 27 system similar to the system shown in FIG. 7 and FIG. 8. The affixing means 24 can include, but is not limited to, hook and loop, adhesive strips, magnetic strips, mortise and tenon, negative air pressure (suction cup or vacuum) device and any means by which the two may be temporarily mated. It will be obvious to one skilled in the art, that the affixing means 29, whereby an attachment (such as a third-party transilluminator or status badge etc.) is affixed to the adapter, can include, but

is not limited to hook and loop, adhesive strips, magnetic strips, mortise and tenon, negative air pressure device and any means by which the two may be temporarily or permanently mated, similarly to the affixing means 24, 31,32,34, 35, 37 disclosed in other figures.

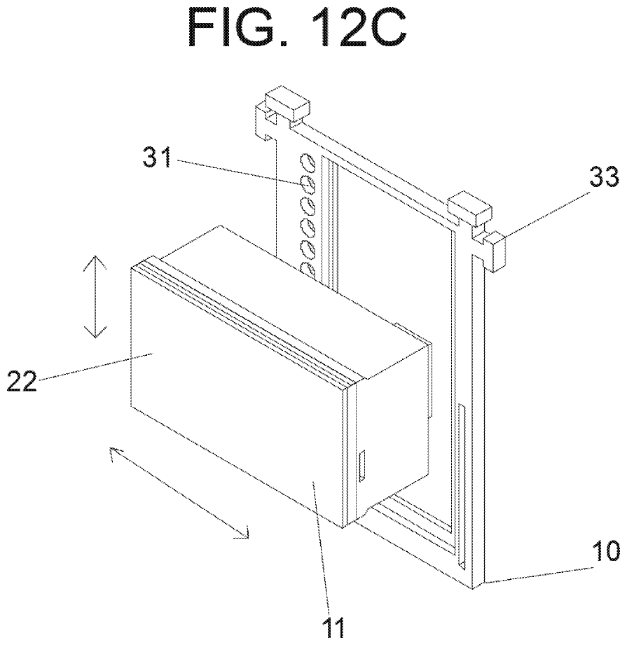

FIGS. 12A-12C disclose an embodiment of the device wherein the affixing means 24, whereby the transilluminating component 11 and the immobilizing component 10 affix to each other comprise an adjustable mortise 31 and tenon 32 (peg and hole) style joint. The placement may be adjusted through the selection of different mortise 31 and tenon 32 combinations. It will be obvious to one skilled in the art, the the size, shape, placement, and orientation of the mortises 31 and tenons 32 may vary. FIG. 12C discloses and embodiment wherein the immobilizing component 10 utilizes tabs 33 and one flexible strap 13 to affix a patient's fingers, which have been curled around the front side of the immobilizer 10 to the bottom and front of the immobilizer 10 and two other straps 13 to affix the patient's arm to the top of the immobilizer 10. FIG. 12C discloses and image wherein the immobilizing component 10 and the transilluminating component 11 have been affixed to each other by inserting a tenon 32 into a mortise 31 and arrows illustrate the possible adjustments to transilluminating component 11 placement via selection of varying mortise 31 and tenon 32 combinations.

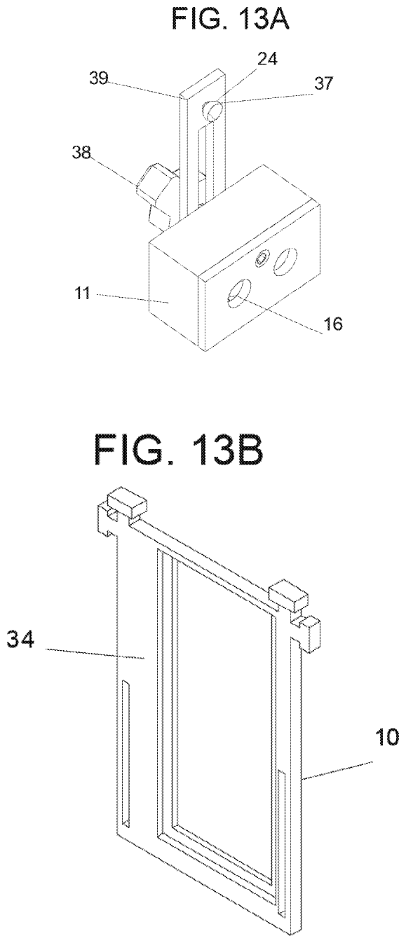

FIGS. 13A-13B disclose an embodiment of the device wherein the affixing means 24, whereby the transilluminator 11 affixes to the immobilizer 10, comprise a suction cup 37 (for affixing to a suction placement surface 34), an adjustment plate 39 (for adjusting the transilluminator 11 placement), and a clamping screw device 38 (for locking the adjustment plate in place).

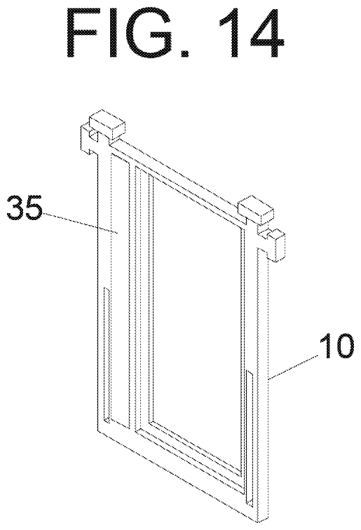

FIG. 14 discloses an embodiment of an immobilizing component similar to that disclosed in FIG. 13B but instead of the suction cup 37 and suction placement surface 34 seen in FIGS. 13A and 13B, an adhesive patch 35 affixes the adjustment plate 39 (FIG. 13A) to the immobilizer 10. It will be obvious to one skilled in the art that the adhesive patch may comprise various types of patches including but not limited to repositionable, reusable, removable, and one-time use chemical adhesives or hook and loop patches. It will also be obvious that the adhesive patch(es) 35 may be located on the transilluminating component 11 rather than the immobilizing component 10 or both (as is necessary with the hook and loop version disclosed).

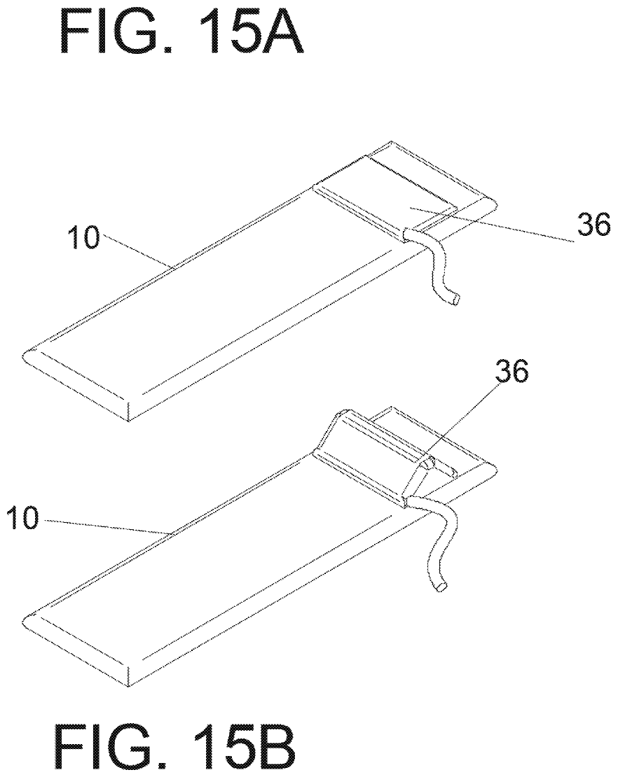

FIGS. 15A and 15B disclose embodiments of an immobilizing component 10 comprising an inflatable bladder 36. The bladder 36 is used instead of a hinge 14 seen in other embodiments to adjust the flexion/extension of an extremity. The bladder is adjusted using fluid power (either liquid or gas). FIG. 15A discloses the bladder 36 in an empty state allowing an extremity to lay flat against the immobilizer 10. FIG. 15B discloses the bladder 36 in a filled (of partially filled) state allowing an extremity to be affixed in a flexed or extended posture (such as the bending of a wrist). In addition to aiding adjustability for patient comfort, adjusted postures work along with affixing means 13 (not shown for improved visualization) to improve the efficacy of the tourniquet effect of affixing means 13. The tourniquet affect, for those unfamiliar, is when outflow of fluid from vessels is restricted. This engorges the vessels and facilitates visualization of a usable vessel and proper needle placement. It will be obvious to those skilled in the art that shapes, orientations, and styles of both bladders 36 and affixing means 13 may vary and still achieve the desired effect.

Advantages of the present invention include the ability for constant immobilization before, during, and after transillumination/IV treatment, as well as compatibility with medical imaging.

In broad embodiment the device is a modular transilluminating immobilizer. One specific example of intended use of the present invention is on an infant patient that is in need of both ongoing intravenous therapy and repeated MRI imaging. In such a situation, the present invention succeeds where current methods and devices fail.

While the aforementioned written description of the invention allows those of ordinary skill to make and use what is considered presently to be the best mode thereof, those of ordinary skill will comprehend and recognize the full worth of the existence of variations, combinations, and equivalents of the specific embodiments, methods, and examples herein. The invention should therefore not be limited by the foregoing described embodiments, methods, and examples, but by all embodiments and methods within the spirit and scope of the invention.

1. A medical device for immobilizing and transilluminating an extremity, the medical device comprising:
an extremity immobilizing component configured to secure to the extremity and immobilize the extremity;
a transilluminating component removably secured to the extremity immobilizing component and configured to provide transillumination of the extremity;
a power source connected to the transilluminating component and configured to provide power thereto; and
an affixing device attached to the extremity immobilizing component and configured to secure the extremity immobilizing component to the extremity;
wherein the medical device affixes to the extremity and provides immobilization of the extremity during use.

2. The medical device of claim 1 wherein the transilluminating component is adjustable with regard to position, direction of light transmission, light frequency and wavelength, and intensity.

3. The medical device of claim 1 wherein the transilluminating component further comprises a plurality of lights.

4. The medical device of claim 1 wherein the transilluminating component can secure to a plurality of positions with respect to the extremity immobilizing component.

5. The medical device of claim 1 wherein the extremity immobilizing component further comprises:
a hinge configured to allow the extremity immobilizing component to pivot.

6. The medical device of claim 1 wherein the extremity immobilizing component further comprises:
a portion utilizing fluid power to change a geometry associated with an extremity contact surface.

7. The medical device of claim 1, wherein the transilluminating component is secured to the extremity immobilizing component via one or more rails extending from the transilluminating component and one or more grooves extending from the extremity immobilizing component.

8. The medical device of claim 1, wherein the transilluminating component is secured to the extremity immobilizing component via one or more mortise and tenon style joints, wherein a protuberance fits securely into a hole or recess.

9. The medical device of claim 1, wherein the transilluminating component is secured to the extremity immobilizing component via one or more sections of adhesive.

10. The medical device of claim 1, wherein the transilluminating component is secured to the extremity immobilizing component via suction (negative air pressure).

11. The medical device of claim 1, wherein the transilluminating component is secured to the extremity immobilizing component via one or more sections of hook and loop closure.

12. The medical device of claim 1 wherein the transilluminating component transmits light in a lateral direction in relation to the extremity.

13. The medical device of claim 1 wherein the transilluminating component is flexible and conforms to contours of a surface upon which the transilluminating component is placed.

14. The medical device of claim 1 wherein the immobilizing component and affixing means are constructed of non-ferromagnetic materials, radiographically, tomographically, and fluoroscopically non-disruptive materials, and materials of no and low magnetic susceptibility.

15. The medical device of claim 1 wherein the transilluminating component is controlled and adjusted wirelessly by a control.

16. The medical device of claim 1 wherein the affixing device comprises one or more straps configured to secure around the extremity.

17. The medical device of claim 1 wherein the extremity immobilizing component is rectangular.

18. The medical device of claim 1 wherein the extremity immobilizing component is transparent.

19. The medical device of claim 1 wherein the transilluminating component transmits light in a parallel direction in relation to the surface of extremity.

20. The medical device of claim 1 further comprising:
an adapter having one or more rails configured to attach to the extremity immobilizing component.

21. The medical device of claim 1, wherein the extremity immobilizing component further comprises:
a hinge that restrains the immobilizing component to a partial range of motion, wherein the range of motion is adjustable.

22. The medical device of claim 1, wherein the transilluminating component further comprises:
a plurality of lights having varying wavelengths.

23. A medical device for immobilizing and transilluminating an extremity, the medical device comprising:
an extremity immobilizing component configured to secure to the extremity and immobilize the extremity;
an adapter component removably secured to the extremity immobilizing component and configured to allow interchangeable use of a plurality of transilluminators which may be removably secured to the adapter;
an affixing device attached to the extremity immobilizing component and configured to secure the extremity immobilizing component to the extremity;
wherein the medical device affixes to the extremity and provides immobilization of the extremity during use.

24. The medical device of claim 24, wherein the adapter component is secured to the extremity immobilizing component via one or more mortise and tenon style joints, wherein a protuberance fits securely into a hole or recess.

25. The medical device of claim 24, wherein the adapter component is secured to the extremity immobilizing component via one or more sections of adhesive.

26. The medical device of claim 24, wherein the adapter component is secured to the extremity immobilizing component via suction (negative air pressure).

27. The medical device of claim 24, wherein the transilluminating component is secured to the extremity immobilizing component via one or more sections of hook and loop closure.

28. A method of transillumination of tissue for venipuncture and vessel access, the method comprising:
affixing an extremity immobilizing component to an extremity of a patient;
adjusting the extremity immobilizing component to a desired posture;
affixing a transilluminating component to the extremity immobilizing component;
positioning the transilluminating component to direct a light in a desired direction;
accessing a vessel of the patient while the extremity is immobilized and while transillumination of the patient is maintained.

29. The method of claim 28, further comprising:
detaching the transilluminating component from the extremity immobilizing component.

30. The method of claim 29, further comprising:
adjusting intensity and color of one or more lights of the transilluminating component.
